(19) United States
(12) Patent Application Publication
(10) Pub. No.: US 2025/0345199 A1
(43) Pub. Date: Nov. 13, 2025

(54) Hallux Valgus Corrector

(71) Applicant: Dongguan Haoxuan Daily Necessities Co., Ltd., Dongguan (CN)

(72) Inventor: Zhixiao Cai, Dongguan (CN)

(21) Appl. No.: 18/898,764

(22) Filed: Sep. 27, 2024

(30) Foreign Application Priority Data
May 8, 2024 (CN) 202420980083.4

(51) Int. Cl. A61F 5/01

(52) U.S. Cl. CPC A61F 5/019

(57) ABSTRACT

A hallux valgus corrector comprises a metatarsal support part, a hallux support part, a hallux wearable part and a sole wearable part. A corrective and adjusting component is arranged on the metatarsal support part for adjusting an angle of the hallux support part. The corrective and adjusting component comprises a second live shaft, a knob, a positioning part and a rope. The sole wearable part is wore on the sole, and the hallux support part is wore on the hallux. Rotate the knob, and the second live shaft rotates and the rope wraps around the second live shaft, thereby pulling the hallux support part to rotate for angle adjustment. Rotate the knob clockwisely or counterclockwisely to adjust the tightness.

### CROSS-REFERENCE TO RELATED APPLICATIONS

The present application claims the benefit of Chinese Patent Application No. 202420980083.4 filed on May 8, 2024, the contents of which are hereby incorporated by reference.

### TECHNICAL FIELD

The present application relates to a technical field of medical equipment, in particular to a hallux valgus corrector.

### BACKGROUND

The existing hallux valgus corrector usually includes a metatarsal support part, a hallux support part, a hallux wearable part arranged on the hallux support part and a sole wearable part arranged on the metatarsal support part. A corrective and adjusting component is assembled on the metatarsal support part to adjust an angle of the hallux support part. A traction end of the corrective and adjusting component is connected on the hallux support part. The metatarsal support part are rotatably connected with the hallux support part through a live shaft. The hallux support part is rotated through the corrective and adjusting component, thereby pulling the hallux to be corrected. Currently, a commonly used method of fixing corrective and adjusting component is rotating buttons on sport shoes to tighten ropes for angle adjustment. This method can only make the adjustment tighter and tighter. If users want to loose it, he/she has to knock the knob for opening and then readjust it, which is inconvenient for adjustment.

### SUMMARY

The present application aims to solve the problem that the corrective and adjusting component of the existing hallux valgus corrector is inconvenient to adjust.

A technical solution adapted to solve the technical problems mentioned in the present application is that: a hallux valgus corrector includes a metatarsal support part, a hallux support part, a hallux wearable part arranged on the hallux support part and a sole wearable part arranged on the metatarsal support part. A corrective and adjusting component is assembled on the metatarsal support part to adjust an angle of the hallux support part. One end of the corrective and adjusting component is connected on the hallux support part. The metatarsal support part includes a metatarsal support body and a connecting part fixedly connected with the metatarsal support body. The corrective and adjusting component is assembled on the connecting part. The hallux support part is rotatably connected with the connecting part through a first live shaft. The corrective and adjusting component includes a second live shaft, a knob, a positioning part and a rope. The second live shaft is rotatably arranged in the connecting part. The knob is fixedly connected with one end of the second live shaft along a radial direction. The positioning part is fixedly connected with the connecting part. One end of the rope is connected with the hallux support part, and other end of the rope is wrapped around the live shaft. An internal gear is arranged on the positioning part. An elastic clamping piece is arranged in the knob to elastically lock with the internal gear. The elastic clamping piece is fixedly connected with the knob along the radial direction.

The technical solutions further limiting the present application include:

The elastic clamping piece includes a left elastic clamping piece and a right elastic clamping piece which are symmetrical to each other. The left elastic clamping piece is connected with the right elastic clamping piece through a connecting arm. The connecting arm is locked in the knob.

The connecting arm is a ring structure with a notch, the left elastic clamping piece and the right elastic clamping piece are located on both sides of the notch on the connecting arm. One end of the second live shaft is provided with a clamping block inserting into the notch.

A support frame is arranged in the knob to support and fix the position of the connecting arm. The connecting arm is locked in the support frame.

The knob is rotatably connected with the positioning part along the radial direction. A knob clamping piece is arranged on the positioning part. A knob locking groove is arranged on the knob to match with the knob clamping piece.

The connecting part is provided with a fixing piece to detachably connect with the connecting part along a vertical direction. The second live shaft and the positioning part are locked between the connecting part and the fixing piece.

A periphery of one end of the second live shaft is provided with a U-shaped locking groove. The clamping block is arranged on the connecting part and the fixing piece to match with the U-shaped locking groove. Other end of the second live shaft is provided with a round positioning plate. The positioning part is provided with a positioning groove to match with the round positioning plate.

The second live shaft is provided with rope fixing holes for fixing the rope.

A swinging end of the hallux support part is provided with a curved threading hole bent toward the connecting part. A first rope guiding hole and a second rope guiding hole for telescopic guidance are arranged on both sides of the hallux support part. One end of the rope penetrates through the first rope guiding hole, the curved threading hole and the second rope guiding hole in sequence. Two ends of the rope are fixedly connected with the second live shaft respectively.

The hallux wearable part is a first tie. Both sides of the hallux support part are provided with first tie mounting holes for mounting the first tie. The first tie is fixed by a first hook-and-loop fastener. The sole wearable part is a second tie. Both sides and a rear side of the metatarsal support part are provided with second tie mounting holes for mounting the second tie. The second tie is fixed by the second hook-and-loop fastener.

By the means of the above technical solutions, the beneficial effects of the present application are: when people use the hallux valgus corrector, the sole wearable part is wore on the sole, the hallux wearable part is wore on the hallux. When the knob is rotated, it drives the second live shaft to rotate, and the elastic clamping piece deforms during rotation. The rotation of the live shaft drives the rope to be wrapped around the second live shaft, thereby pulling the hallux support part to rotate for angle adjustment. When the knob is stopped to rotate, the elastic clamping piece is restored to lock with the internal gear on the positioning part, so that the hallux support part and the metatarsal support part are relatively fixed. Rotate the knob clockwisely or counterclockwisely to adjust the tightness. It is simple and convenient to adjust.

### BRIEF DESCRIPTION OF DRAWINGS

FIG. 1 is a schematic diagram of the three-dimensional structure of the hallux valgus corrector of the present application;

FIG. 2 is a schematic diagram of the cross-sectional structure of the corrective and adjusting component of the hallux valgus corrector thereof;

FIG. 3 is a schematic diagram of the exploded structure of the hallux valgus corrector thereof;

FIG. 4 is a schematic diagram of the exploded structure of the hallux valgus corrector from another direction;

FIG. 5 is a structure diagram of the knob of the hallux valgus corrector.

Reference signs in the drawings:
1. metatarsal support part; 11. metatarsal support body; 12. connecting part; 121. fixing piece; 2. hallux support part; 21. curved threading hole; 22. first rope guiding hole; 23. second rope guiding hole; 24. first tie mounting hole; 3. hallux wearable part; 4. sole support part; 5. corrective and adjusting component; 51. second live shaft; 511. clamping block; 512 U-shaped locking groove; 513. round positioning plate; 514. rope fixing hole; 52. knob; 521. elastic clamping piece; 5211. left elastic clamping piece; 5212. right elastic clamping piece; 5213. connecting arm; 522. support frame; 5221. support rib part; 5222. clamping rib part; 53. positioning part; 531. internal gear; 532. knob clamping piece; 533. positioning groove; 534. positioning fastener; 54. rope; 6. first live shaft.

### DESCRIPTION OF EMBODIMENTS

The structure of the present application is further described below in conjunction with the figures.

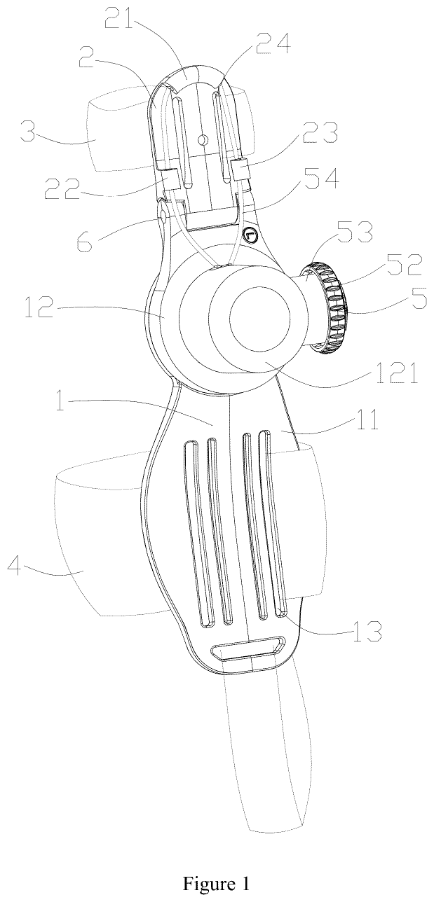

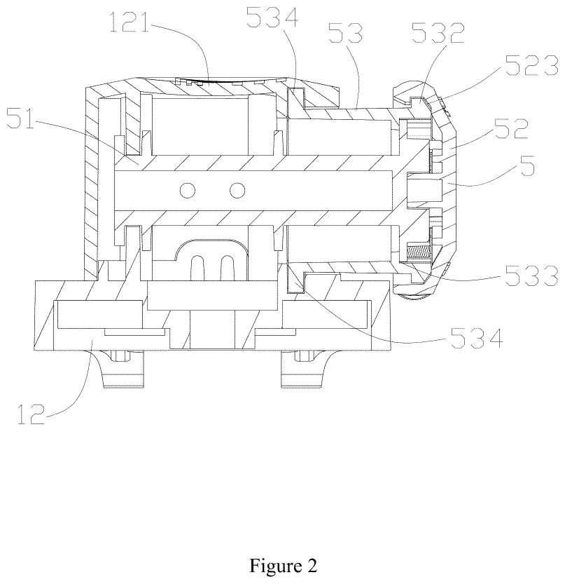

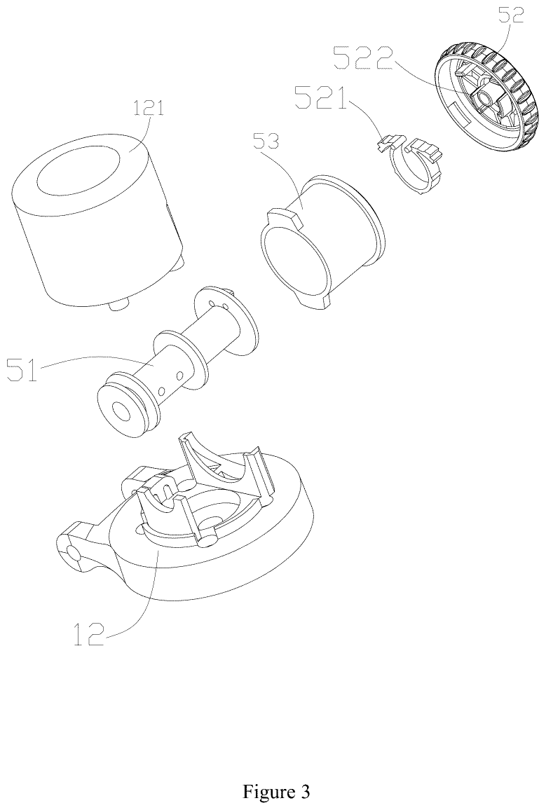

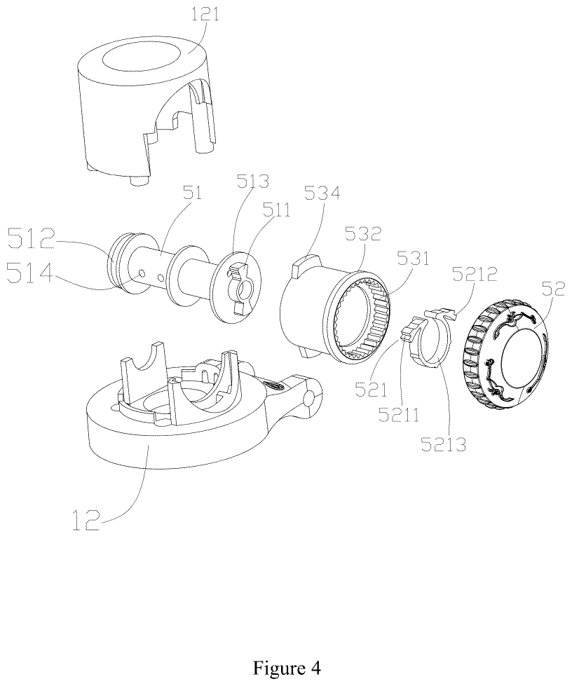

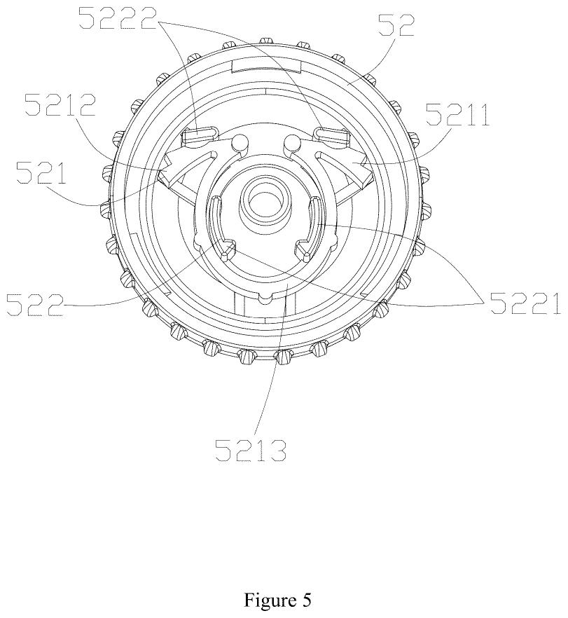

Referring to FIGS. 1 to 5, the hallux valgus corrector includes a metatarsal support part 1, a hallux support part 2, a hallux wearable part 3 arranged on the hallux support part 2 and a sole support part 4 arranged on the metatarsal support part 1. A corrective and adjusting component 5 is assembled on the metatarsal support part 1 to adjust an angle of the hallux support part 2. One end of the corrective and adjusting component 5 is connected on the hallux support part 2. The metatarsal support part 1 includes a metatarsal support body 11 and a connecting part 12 fixedly connected with the metatarsal support body 11. The corrective and adjusting component 5 is assembled on the connecting part 12. The hallux support part 2 is rotatably connected with the connecting part 12 through a first live shaft 6. The corrective and adjusting component 5 includes a second live shaft 51, a knob 52, a positioning part 53 and a rope 54. The second live shaft 51 is rotatably arranged in the connecting part 12. The knob 52 is fixedly connected with one end of the second live shaft 51 along a radial direction. The positioning part 53 is fixedly connected with the connecting part 12. One end of the rope 54 is connected with the hallux support part 2, and other end of the rope is wrapped around the live shaft. An internal gear 531 is arranged on the positioning part 53. An elastic clamping piece 521 is arranged in the knob 52 to elastically lock with the internal gear 531. The elastic clamping piece 521 is fixedly connected with the knob 52 along the radial direction. When in use, the sole wearable part 4 is wore on the sole, the hallux wearable part 3 is wore on the hallux. When the knob 52 is rotated, the knob 52 drives the second live shaft 51 to rotate, so that the elastic clamping piece 521 deforms during rotation. The rotation of the live shaft drives the rope 54 to be wrapped around the second live shaft 51, thereby pulling the hallux support part 2 to rotate for angle adjustment. When the knob 52 is stopped to rotate, the elastic clamping piece 521 is restored to lock with the internal gear 531 on the positioning part 53, so that the hallux support part 2 is relatively fixed with the metatarsal support part 1. Rotate the knob clockwisely or counterclockwisely to adjust the tightness. It is simple and convenient to adjust or readjust.

In this embodiment, the elastic clamping piece 521 includes a left elastic clamping piece 5211 and a right elastic clamping piece 5212 which are symmetrical to each other. The left elastic clamping piece 5211 is connected with the right elastic clamping piece 5212 through a connecting arm 5213. The connecting arm 5213 is locked in the knob 52. When the knob 52 is rotating, it drives the connecting arm 5213 to rotate, and the elastic clamping piece 5211 and the right elastic clamping piece 5212 deform simultaneously. When the knob 52 is stopped to rotate, the left elastic clamping piece 5211 and the right elastic clamping piece 5212 are restored and locked with the internal gear 531 to fix the position of the knob 52, thereby fixing the position of the second live shaft 52, and making the hallux support part 2 and the metatarsal support part 1 relatively fixed, which makes the adjustment simple and convenient. When adjusting, the symmetrical left elastic clamping piece 5211 and right elastic clamping piece 5212 deform elastically with the inner gear 531, so that they slide and abut against each other, which makes the adjustment stable.

In this embodiment, the connecting arm 5213 is a ring structure with a notch. The left elastic clamping piece 5211 and the right elastic clamping piece 5212 are located on both sides of the notch on the connecting arm 5213. One end of the second live shaft 51 is provided with a clamping block 511 inserting into the notch. When the knob 52 is rotated to drive the connecting arm 5213 to rotate, the connecting arm 5213 drives the second live shaft 51 to rotate

In this embodiment, a support frame 522 is arranged in the knob 52 to support and fix the position of the connecting arm 5213. The connecting arm 5213 is locked in the support frame 522. The support frame 522 includes a support rib part 5221 for supporting the internal wall of the connecting arm 5213, and a clamping rib part 5222 to lock the left elastic clamping piece 5211 and the right elastic clamping piece 5212 and prevent the connecting arm 5213 from rotating. The support rib part 5221 prevents the connecting arm 5213 from deforming, and the clamping rib part 5222 prevents the connecting arm 5213 from rotating.

In this embodiment, the knob 52 is rotatably connected with the positioning part 53 along the radial direction. The positioning part 53 is provided with knob clamping pieces 532. The knob 52 is provided with knob locking grooves 523 for matching with the knob clamping pieces 532. The knob 52 is rotatably locked with the positioning part 53, thereby fixing the position of the knob 52 along an axial direction.

In this embodiment, a fixing piece 121 is arranged on the connecting part 12 for detachably connecting with the connecting part 12 along a vertical direction. The second live shaft 51 and the positioning part 53 are locked between the connecting part 12 and the fixing piece 121. An upper end and a lower end of the positioning part 53 are provided with positioning fasteners 534. The positioning part 53 is locked with the fixing piece 121 and the connecting part 12 through the positioning fasteners 534. A periphery of one end of the second live shaft 51 is provided with a U-shaped locking groove 512. Both the connecting part 12 and the fixing piece 121 are provided with clamping blocks 511 for matching with the U-shaped locking groove 512. Other end of the second live shaft 51 is provided a round positioning plate 513. The positioning part 53 is provided with a positioning groove 533 to match with the round positioning plate 513. When the second live shaft 51 is rotated, two ends of the second live shaft 51 are fixed the position with the positioning groove 533 through the U-shaped locking groove 512, so that the second live shaft 51 is capable of rotating stably.

In this embodiment, the second live shaft 51 is provided with rope fixing holes 514 to fix the rope 54. The rope fixing holes 514 are used for fixing the rope 54.

In this embodiment, a swinging end of the hallux support part 2 is provided with a curved threading hole 21 bent toward the connecting part 12. Two sides of the hallux support part 2 are provided with a first rope guiding hole 22 and a second rope guiding hole 23 for telescopic guidance. One end of the rope 54 penetrates through the first rope guiding hole 22, the curved threading hole 21 and the second rope guiding hole 23 in sequence. Two ends of the rope 54 are fixedly connected with the second live shaft 51 respectively. The rope fixing hole 514 is provided with two. Two ends of the rope 54 are fixed and tied with two rope fixing holes 514 respectively.

In this embodiment, the hallux wearable part 3 is a first tie. Two sides of the hallux support part 2 are provided with first tie mounting holes 24 for mounting the first tie. The first tie is fixed by a first hook-and-loop fastener. The sole wearable part is a second tie, two sides and a rear side of the metatarsal support part 1 are provided with second tie mounting holes 13 for mounting the second tie. The second tie is fixed by the second hook-and-loop fastener.

Although the specific implementation of the present application is described in detail in conjunction with the figures, it should not be understood as limiting the protection scope of the present application. Within the scope described in the claims, various modifications and variations that can be made by those skilled in the art without creative work still belong to the protection scope of the present application.

1. A hallux valgus corrector, comprises a metatarsal support part, a hallux support part, a hallux wearable part arranged on the hallux support part, and a sole wearable part arranged on the metatarsal support part; a corrective and adjusting component is assembled on the metatarsal support part to adjust an angle of the hallux support part; one end of the corrective and adjusting component is connected on the hallux support part;
wherein the metatarsal support part comprises a metatarsal support body and a connecting part fixedly connected with the metatarsal support body; the corrective and adjusting component is assembled on the connecting part; the hallux support part is rotatably connected with the connecting part through a first live shaft;
the corrective and adjusting component comprises a second live shaft, a knob, a positioning part, and a rope, wherein the second live shaft is rotatably arranged in the connecting part; the knob is fixedly connected with one end of the second live shaft along a radial direction; the positioning part is fixedly connected with the connecting part; one end of the rope is connected with the hallux support part, and other end of the rope is wrapped around the live shaft; an internal gear is arranged on the positioning part; an elastic clamping piece is arranged in the knob to elastically lock with the internal gear; the elastic clamping piece is fixedly connected with the knob along the radial direction.

2. The hallux valgus corrector according to claim 1, wherein the elastic clamping piece comprises a left elastic clamping piece and a right clamping piece which are symmetrical to each other; the left elastic clamping piece and the right elastic clamping piece are connected through a connecting arm; the connecting arm is locked in the knob.

3. The hallux valgus corrector according to claim 2, wherein the connecting arm is a ring structure with a notch; the left elastic clamping piece and the right elastic clamping piece are located on both sides of the notch on the connecting arm; one end of the second live shaft is provided with a clamping block inserting into the notch.

4. The hallux valgus corrector according to claim 2, wherein the knob is provided with a support frame to support and fix the position of the connecting arm; the connecting arm is locked in the support frame.

5. The hallux valgus corrector according to claim 1, wherein the knob is rotatably connected with the positioning part along the radial direction; the positioning part is provided with a knob clamping piece; the knob is provided with a knob locking groove to match with the knob clamping piece.

6. The hallux valgus corrector according to claim 1, wherein the connecting part is provided with a fixing piece to detachably connect with the connecting part along a vertical direction; the second live shaft and the positioning part are locked between the connecting part and the fixing piece.

7. The hallux valgus corrector according to claim 6, wherein a U-shaped locking groove is provided around one end of the second live shaft's periphery; the clamping block is arranged on the connecting part and the fixing piece to match with the U-shaped locking groove; other end of the second live shaft is provided with a round positioning plate; the positioning part is provided with a positioning groove to match with the round positioning plate.

8. The hallux valgus corrector according to claim 1, wherein the second live shaft is provided with rope fixing holes to fix the rope.

9. The hallux valgus corrector according to claim 1, wherein one end of the hallux support part is provided with a curved threading hole bent toward the connecting part; a first rope guiding hole and a second rope guiding hole for telescopic guidance are arranged on both sides of the hallux support part; one end of the rope penetrates through the first rope guiding hole, the curved threading hole, and the second rope guiding hole in sequence; two ends of the rope are fixedly connected with the second live shaft respectively.

10. The hallux valgus corrector according to claim 1, wherein the hallux wearable part is a first tie; both sides of the hallux support part are provided with first tie mounting holes for mounting the first tie; the first tie is fixed by a first hook-and-loop fastener; the sole wearable part is a second tie; both sides and a rear side of the metatarsal support part are provided with second tie mounting holes for mounting the second tie; the second tie is fixed by the second hook-and-loop fastener.
